(19) United States
(12) Patent Application Publication
(10) Pub. No.: US 2007/0031442 A1
(43) Pub. Date: Feb. 8, 2007

(54) Method and compositions for boosting immune response

(76) Inventor: Andrew Sewell, Oxford (GB)

Correspondence Address: BELL & ASSOCIATES, 416 FUNSTON ST., SUITE 100, SAN FRANCISCO, CA, 94118, US

(21) Appl. No.: 10/545,580

(22) PCT Filed: Feb. 17, 2004
(86) PCT No.: PCT/GB04/00623

§ 371 Date: Feb. 27, 2006

(30) Foreign Application Priority Data
Feb. 17, 2003 (GB) 0303645.6

(51) Int. Cl. A61K 39/00; C07H 21/04; C12P 21/06; C07K 14/74; C07K 14/16

(52) U.S. Cl. 424/185.1; 530/350; 435/69.1; 435/320.1; 435/325; 514/12; 536/23.5

(57) ABSTRACT

An antigen presenting molecule is capable of increasing sensitivity of a cytotoxic T lymphocyte to an antigen by interaction with a T cell receptor and CD8 coreceptor thereon, when the antigen presentation molecule displays an increased avidity for the CD8, compared to the molecule encoded by SEQ ID NO. 1 in vivo. Levels of avidity of the molecule for CD8 in excess of the pMHC I/TCR interaction result in the molecule being a pan activator for MHC Class I specific cytotoxic T cells.

The present invention relates to an antigen presentation molecule, and uses thereof, in the treatment of disease in an individual.

Viral infection and tumours are major causes of disease in the human or animal body. Both of these present a particular problem for the immune system, since the target antigens, by which the immune system recognises the virus or tumour, are hidden within autologous cells. This problem has, for the most part, been overcome by the major histocompatibility complex (MHC) Class I antigen presentation pathway, also known as the endogenous pathway.

Essentially, this well characterised pathway indiscriminately takes intracellular viral and autologous peptides and transports them to the cell surface. Proteins are first degraded in the cytosol into peptides of roughly 8 to 11 amino acids in length. Once these peptides have been transported into the endoplasmic reticulum, they are bound by MHC Class I proteins and are presented at the cell surface, where the MHC Class I molecules act as antigen presentation molecules.

Thus, cells display a selected peptide library from all of their expressed genes on their cell surface, accessible to immune surveillance. Most of the autologous peptides presented on the cell surface are recognised as “self” and are, therefore, ignored by the immune system.

However, some peptides result from the expression of altered genes or dysregulated expression of the individual's genes, as can occur in cancer. These altered or dysfunctional peptides, as well as any virally-encoded peptides, are also presented on the cell surface, complexed with MHC Class I.

These “non-self” or foreign-peptide/MHC Class I complexes are recognised by circulating CD8⁺ cytotoxic T lymphocytes (CTL). This recognition leads to intracellular signalling and activation of the CTL. Once activated, these white blood cells eliminate the cell which has been identified as having presented non-self peptides. In addition, the activated CTL releases soluble factors which communicate this recognition event to the rest of the immune system.

The antigen specificity of CTL's is conferred by the T cell receptor (TCR), whose highly variable complementarity determining regions interact with the a α1/α2 domain platform of the MHC Class I molecule incorporating the foreign peptide. In addition to its role in presenting the antigen to the TCR, the peptide-MHC Class I molecule complex (pMHCI) also interacts with the CTL co-receptor, CD8, which binds to the invariable regions of the MHC Class I α3 domain. The CD8 co-receptor is critical for both development and activation of CTL's. It has been proposed that CD8 performs a role in signal transduction, assists cell-cell adhesion and cooperatively enhances the TCR/pMHCI interaction (Gao, et al. 2000; Immunology Today 21 no. 12:630).

The effect of blocking the pMHC/CD8 interaction on the activation of CD8⁺ T cells has been investigated. Sewell, et al. 1999 (Nat. Med. 5, no. 4:399) showed that a soluble form of the CD8 co-receptor can block activation of CD8⁺ T cells. While full activation and proliferation of a CD8⁺ T cell requires both the TCR/pMHCI and the pMHC/CD8⁺ interactions, the activation induced cell death of a target cell has been shown to be completely independent of the pMHCI/CD8 interaction (Xu, et al. 2002 Immunity 14, no. 5:591).

Purbhoo et al. 2001 (J. Biol. Chem. 276, no. 35:32786) showed that the pMHCI/CD8 interaction results in the complete phosphorylation of the TCR ζ chain. This was demonstrated by decreasing or abrogating the pMHCI/CD8 interaction by mutation of the pMHCI molecule. The multistep phosphorylation of all six tyrosine residues in the three immunoreceptor tyrosine activation motifs (ITAMs) of the TCR ζ chain is essential for T cell activation (Kersh, et al. 1988, Science 281, no. 5376:572). Purbhoo, et al. (supra) showed that the degree of phosphorylation of the TCR ζ chain is directly correlated to the affinity of the pMHCI/CD8 interaction. TCR engagement in the absence of a pMHCI/CD8 interaction results in preferential induction of partially phosphorylated CD3ζ (p21 phosphoform) and so cannot effect T cell activation at physiological levels of antigen. The cytoplasmic domains of CD8 associate with the intracellular protein-tyrosine kinase p56^{lck} which is critical for the initiation of CTL signal transduction. Inefficient recruitment of p56^{lck} to the TCR/CD3 complex in the absence of a pMHCI/CD8 interaction is thought to explain the incomplete phosphorylation of CD3ζ.

Despite increased understanding of the immune response to endogenous non-self peptides, viral infections can still lead to serious disease and even death. Furthermore, tumours, although sometimes treatable, can also frequently lead to death, especially if the tumour is not caught at an early stage.

Due to the enormous number of potential foreign or altered peptides that may be presented at the surface of a cell, the immune system may often have only one CTL line bearing TCR's that recognise the altered or foreign-peptide MHC I complex. When this CTL recognises the pMHCI complex, soluble factors are released to alert the rest of the immune system. These soluble factors include, for example, the chemotactic cytokines (or chemokines), MIP1β and RANTES, which are involved in leukocyte activation. The level of response by the immune system to these signals is controlled by the level of chemokines release.

Therefore, if the activation signal arising from the CTL/Jantigen presentation cell interaction is weak, then the immune response will also be weak. For instance, MIP1β can induce the proliferation and activation of killer cells. However, if little MIP1β is released, then the chemotactic and activating effects of this chemokine will be reduced, with the result that fewer leukocytes will be activated or attracted to the area of infection or tumour activity.

The use of adjuvants tends to be non-specific, generally causing a heightened reaction to anything administered in conjunction with the adjuvant. In addition, it is not always desirable to heighten the sensitivity of the immune system when what is required is to heighten the response to a specific antigen.

WO02/46399 discloses recombinant MHC1 with increased affinity for peptide ligand. Affinity for CD8 is not discussed.

U.S. Pat. No. 6,153,408 discloses altered MHCI and MHCII molecules so that the domains are covalently linked.

Connolly et al. (PNAS vol. 87, 1990, pp2137-2141) disclose that residues 222-229 are important for CD8 recognition and that, if altered, lead to a decrease in CD8 recognition by the MHC1 molecule.

Gao et al (J. Biol Chem Vol. 275, 2000, pp. 15232-15238) disclose the differing binding affinities of various MHC1 alleles for CD8αα.

Glick et al. (J. Biol Chem Vol. 277, 2002, pp. 20840-20846) disclose that mutation of lysine 58 leads to a decrease in CD8 binding by MHC1.

Maeurer et al. (Int. J. Cancer, Vol. 97, 2002, pp. 64-71) disclose ways to expand or optimise identification and expansion of antigen specific T cells.

Surprisingly, it has now been found that increasing the pMHCI/CD8 interaction by mutation of the CD8 binding region of the MHC Class I molecule leads to an increase in the effector response of the CTL to a given quantity of antigen.

Thus, in a first aspect, the present invention provides an antigen presenting molecule capable of activating a cytotoxic T lymphocyte by interaction with a T cell receptor and CD8 coreceptor thereon, wherein the antigen presentation molecule displays an increased avidity for the CD8 coreceptor, compared to the molecule encoded by SEQ ID NO. 1 in vivo.

The antigen presenting molecule of the invention generally corresponds closely to MHC I, and is preferably derived directly therefrom. In particular, it is preferred to provide the antigen-binding, TCR-binding and CD8-binding domains of MHC L these domains corresponding substantially closely to human MHC I in order to avoid non-self problems. This applies, even where the antigen presentation molecule is produced in another species, such as mouse, rat, rabbit or sheep, for example.

The antigen presentation molecule of the invention binds CD8 with greater avidity than normal MHC I. This may be achieved by suitable sequence alteration of the molecule in order to enhance the interaction. It has been found that this is generally best achieved by amendment of the residues that interact directly with the CD8 molecule, but it is also possible to enhance other residues in the binding area to add side chains with greater affinity for residues on CD8, for example.

The antigen presentation molecule is preferably soluble, and soluble MHC I is described in O'Callaghan et al. (Anal. Biochem. (1999), 266, no. 1:9). There is no restriction on the antigen presentation molecule, provided that it serves to bind TCR and CD8 to initiate the effector response. Where the molecule is not soluble, then it may be bound to a suitable carrier, such as a liposome, for distribution in vivo.

The antigen presenting molecule of the invention will generally be referred to as MHC I of the invention, or just MHC I, but it will be understood that such reference includes reference to all molecules of the invention, regardless of species, for example.

MHCI of the invention, such as the HLA A⁺ mutant, may be used, as indicated above, as soluble multimers, such as tetramers, or may be expressed as the full-length molecule in cells previously engineered to be MHC I deficient. For example, HLA A2 EBV transformed B cell transfectants are envisaged in MHC I-deficient cells. HLA A+ is used herein to refer to Human Leukocyte Antigen A*0201 with the Q115E substitution. HLA A+ is a particularly preferred molecule of the invention, as it has increased avidity for CD8 without approaching levels associated with the MHC/TCR interaction, so that sensitivity for any antigenic fragment associated therewith is substantially increased, without affecting specificity of the CTL for the antigen.

The level of binding to CD8 should be greater than that which occurs normally, but not so great as to unbalance the APC/TCR interaction. Typical TCR/antigen interaction has a K_{D} of 1-50 μM, and it is preferred that the avidity of the altered MHCI-CD8 interaction falls within this range. In particular, it is preferred that this range is not exceeded. Furthermore, it is generally preferred that the increase in avidity for CD8 should not greatly exceed 10 fold, with a level of between 2 to 8 fold being more preferable. This has the advantage of increasing the CTL response to the antigen, without affecting the specificity of the response, thereby avoiding the likelihood of eliciting a false positive response. In general, it is preferred that antigen presentation molecules of the present invention elicit no more false responses than naturally occurring MHC I.

It is generally preferred that the antigen presentation molecule of the invention is bound to the antigen against which it is desired to boost the immune response. This binding may simply rely on the natural avidity of MHC I for the antigen. Alternatively, a suitable means for ensuring the binding may be employed. For instance, this can be done by linking the peptide antigen to the rest of the molecule, for example, by linking it to β2 microglobulin during expression (Tafuro S., et al (2001), Eur. J. Immunol. 31:440-9) or by other means known to the skilled person. Alternatively, linking may be achieved by expressing the peptide and MHC I together in a suitable expression system.

It is also envisaged that an antigen presentation molecule of the invention may be expressed in such a configuration that the TCR is bound as though the antigen presentation molecule were actually bound to the relevant antigen. This is not generally especially commercially feasible, but may be desirable where an antigen is especially toxic or otherwise dangerous, for example.

It is a particular advantage of the present invention that it may be used to boost a low-level immune response in an individual. This is particularly advantageous in combating viral infections or in stimulating the body to respond to, and preferably destroy, tumours.

Therefore, although this mutation has no effect on the antigen specificity of the CTL, once an antigenic peptide is identified by a CTL, through the formation of the pMHCI/TCR complex, the increased pMHCI/CD8 interaction, resulting from the mutation of the MHC Class I molecule, provides for an increased response by the CTL.

The antigen presentation molecule of the invention may be of particular use in the treatment of tumours, as cancers are less likely to provide selective pressure on a population, and generally are associated with antigens eliciting a lesser response than those associated with foreign organisms. Boosting the response to tumour antigens then provides a significant weapon with which to fight this disease.

### FIGURE LEGEND AND COMMENTS

FIG. 1 shows that mutation of the MHC class I heavy chain α3 domain can increase binding to CD8. Mutation of the α3 domain does not affect the binding of the TCR to the α1/α2 peptide-binding platform of MHC class I molecules (Purbhoo et al, 2001 supra).

FIG. 1A shows the surface plasmon resonance-measured binding of CD8 to wild type HLA A*68011, 227/8KA mutated HLA A*068011 and 245A substituted HLA A*068011. The 245A substitution in HLA A*68011 increases the interaction between A68 and CD8 by almost 10 fold. In HLA A68 the bulky valine at position 245 leads to a distortion of the α3 domain loop (223-229) important for CD8 binding. This point mutation was previously thought to prevent binding to CD8. However, we have shown that HLA A68 interacts weakly with CD8 (K_{D}=980 μM) and that despite being weak this interaction maintains biological significance. If the valine at position 245 is mutated to an alanine, then the strength of the HLA A68/CD8 interaction is increased by 9 fold (K_{D}=102 μM).

FIG. 1B shows that a Q115->E, and a Q115->E and T225->Y double substitution, increase the interaction of HLA A*0201 with CD8αα. Kd values are an average of three experiments. Standard deviation from the mean is shown.

FIG. 2 shows that targets bearing HLA A*68011 molecules with increased CD8-binding act as significantly better antigen presenting cells.

FIG. 2A shows interferon γ ELISpot using HLA A68011-restricted HIV-1 Tat-specific T cells (Oxenius, A et al, 2002, Aids 16, no. 9:1285) and antigen pulsed C1R bearing equal amounts of wild type HLA A*68011, CD8 ‘null’ HLA A*6801 and HLA A*6801 with increased CD8 binding as antigen-presenting targets (245V). C1R B cell lines expressing either A68, A68 DT227/8KA or A68 V245A were produced and used to present peptide (ITKGLGISYGR: SEQ ID NO. 11) to a HIV-1 Tat restricted CTL clone c23. CTL activated at significantly lower peptide concentrations (10 fold lower) when peptide was presented in the context of the V245A mutation compared to wild type presentation. The % of CTL activating at a given peptide concentration increased by up to 30% when peptide was presented in the context of an increased pMHCI/CD8 interaction.

In FIG. 2B, the above targets were used to present antigen to HLA A68011-restricted, HIV Tat-specific T cells (Oxenius et al, 2001, supra). Supernatant was assayed for the presence of RANTES as described previously.

FIG. 2C is as for FIG. 2B, but supernatants were assayed for MIP-1β (Purbhoo et al, 2001 supra).

FIG. 3 shows that the presentation of antigen in the context of mutant HLA A*68011 with increased CD8-binding does not alter T cell specificity. C1R targets bearing equal amounts of wild type HLA A*68011, CD8 ‘null’ HLA A*68011 and HLA A*68011 with increased CD8 binding (245A) were pulsed with A68-Tat epitope and natural variants of this epitope. These cells do not recognise any of the hundreds of self peptides complexed with HLA A68 on the surface of these cells as they do not activate without the addition of antigen, even in the presence of increased pMHCI/CD8 interaction. The pattern of recognition of variants remains the same with each target. Increased pMHCI/CD8 interaction results in better recognition of the weak agonist 4A peptide.

FIG. 4 shows that CTL antigen sensitivity and effectivity is enhanced by increasing the HLA A2/CD8 interaction without any loss of specificity.

FIG. 4A shows that HLA A2 restricted CTL specific for either Tyrosinase (YMDGTMSQV) or HIV-1 Gag (SLYNTVATL) activate at significantly lower peptide concentrations if peptide is presented in the context of an increased pMHCI/CD8 interaction. The production of MIP-1β by 2 HIV-1 Gag specific (SLYNTVATL) clones was enhanced by up to 3 fold in response to peptide presented in the context of an increased pMHCI/CD8 interaction.

FIG. 4 B is an Interferon γ Elispot using the A*0201 SLYNTVATL (HIV-1 Gag) specific clone 003. The pattern of recognition of naturally occurring HIV-1 Gag peptide variants by this CTL clone is not altered when they are presented in the context of an increased pMHCI/CD8 interaction. Increasing the pMHCI/CD8 interaction enhances CTL sensitivity to the index peptide and to a lesser degree to the weak agonists (3H and 3S). Presentation of an antagonist in the context of the HLA A*0201 Q115E mutant (K_{D}=85 μM) does not result in CTL activation.

FIG. 5 demonstrates that increasing the pMHCI/CD8 interaction mediates enhanced phosphorylation of TCR associated CD3ζ, which is the biochemical basis for the observed enhancement in T cell sensitivity and effectivity.

FIG. 5A shows that wild type and HLA A*0201 tetramers stained 003 CTL efficiently and at the same intensity at different tetramer concentrations.

FIG. 5B Anti-phosphotyrosine immunoblots demonstrate that cross-linking of the TCR in the absence of a pMHCI/CD8 interaction fails to induce phosphorylation of the CD3ζ chain. Wild type tetramers induce p23 formation which is the fully phosphorylated form of the CD3ζ chain required for T cell activation. Stimulating 003 CTL with HLA A+/SLYNTVATL tetramer gives increased levels of p23 relative to the incompletely phosphorylated form p21 in comparison to 003 stimulated by wild type tetramer.

FIG. 6 shows that the enhancement of T cell sensitivity and effectivity provided by pMHCI with increased CD8-binding extends to soluble forms of antigen.

FIG. 6A shows multimeric forms of wildtype and mutated forms of HLA A2 stain cells bearing a relevant TCR with equal intensity.

FIG. 6B shows multimeric forms of HLA A2, with increased CD8 binding, induce significantly better responses from antigen-specific T cells. Tetrameric forms of the peptide SLYNTVATL (SEQ ID NO. 7) bound to HLA A2 were made and used to activate either the CTL clone 5C11 or 5C3. Activation was measured by MIP1-βproduction.

FIG. 6C is as for FIG. 6B, but using multimeric HLA A*68011 to stimulate A68011-restricted HIV-1 Tat-specific T cells (Oxenius et al, 2001, supra).

FIG. 7 shows that soluble pMHCI with a super-enhanced ability to bind to CD8 can stain all CD8+ T cells and activate them in an antigen independent manner, thereby providing a MHC class I-specific superantigen.

FIG. 7A shows that the human pMHCI/CD8 interaction is characterised by low affinity (K_{D}=128 μM). pMHCI tetramer folded around SLYNTVATL or LLFGYPVYV peptide stained 0.73% and 1% of CD3+ CD8+PBMC respectively. Introducing the Kb mutation into pMHCI tetramers folded around SLYNTVATL or LLFGYPVYV peptide increases this staining to 85.3% and 83.73% respectively. Therefore, increasing the affinity of the CD8/pMHCI interaction by approximately 25 fold, so that its strength approaches that of the TCR/pMHCI interaction, results in a total loss of tetramer staining specificity.

FIG. 7B shows that wild type MHCI tetramer folded around the SLYNTVATL peptide stained 003 CTL well, whereas wild type pMHCI tetramer folded around an irrelevant Tax peptide (LLFGYPVYV) did not stain. Therefore, pMHCI tetramers with ‘normal’ pMHCI/CD8 interaction stain CTL specifically. pMHCI tetramers folded around SLYNTVATL or irrelevant peptide will both stain 003 CTL, if the A2 Kb mutation is introduced. In the A2Kb mutation, the α3 domain of human HLA A2 is substituted with the α3 domain of murine H-2K^{b}, thereby producing a chimeric class I, designated A2Kb. This mutation increases the pMHCI/CD8 interaction by 25 fold. Therefore, increasing this interaction gives a reagent that will stain all CTL independently of a specific TCR/pMHCI interaction.

FIG. 7C shows that A2 SLYNTVATL and A2 M SLYNTVATL tetramer activated 003 CTL well, producing all 3 lymphokines. A2 LLFGYPVYV did not activate 003 CTL. Therefore, CTL activation by tetramer with ‘normal’ pMHCI/CD8 interaction maintains specificity. A2 A2Kb LLFGYPVYV resulted in significant production of MIP-1β, RANTES and IFNγ, above background, which suggests that increasing the pMHCI/CD8 interaction so that it becomes greater than the TCR/pMHCI interaction can result in CTL activation in the absence of specific peptide recognition.

FIG. 8 is a model of the key interactions in T cell activation.

FIG. 8A shows that the TCR provides the specificity for interaction and ensures that only certain pMHCI molecules are recognised.

FIG. 8B shows that the coreceptor governs the amount of intracellular signalling generated and, thus, the magnitude of effector function.

FIG. 8C shows the effects of mutating pMHCI to slightly increase (K_{D}=85 μM) or decrease the interaction of CD8 with pMHCI. Increasing the coreceptor binding slightly need not alter T cell specificity but enhances the sensitivity of T cells and increases the magnitude of their effector function in response to a given quantity of antigen. This can be capitalised on to specifically increase the T cell response to any antigen of choice.

FIGS. 9 and 10 are molecular models.

FIG. 9 shows the relationship between HLA-A2 altered residue Q115E and CD8 αl R4 residue.

FIG. 10 shows the relationship between HLA-A2 altered residue T225V and various CD8 α1 and α2 residues.

### SEQUENCE LISTING INDEX

SEQ ID NO. 1 is the nucleotide sequence encoding the human MHC Class I HLA-A*020101 allele.

SEQ ID NO. 2 is the protein sequence encoded by the human MHC Class I HLA-A*020101 allele.

SEQ ID NO. 3 is the nucleotide sequence of human MHC Class I HLA-A*680101 allele.

SEQ ID NO. 4 is the protein sequence encoded by the human MHC Class I HLA-A*680101 allele.

SEQ ID NO. 5 is the nucleotide sequence of human MHC Class I HLA-B*4801 allele.

SEQ ID NO. 6 is the protein sequence encoded by the human MHC I HLA-B*4801 allele.

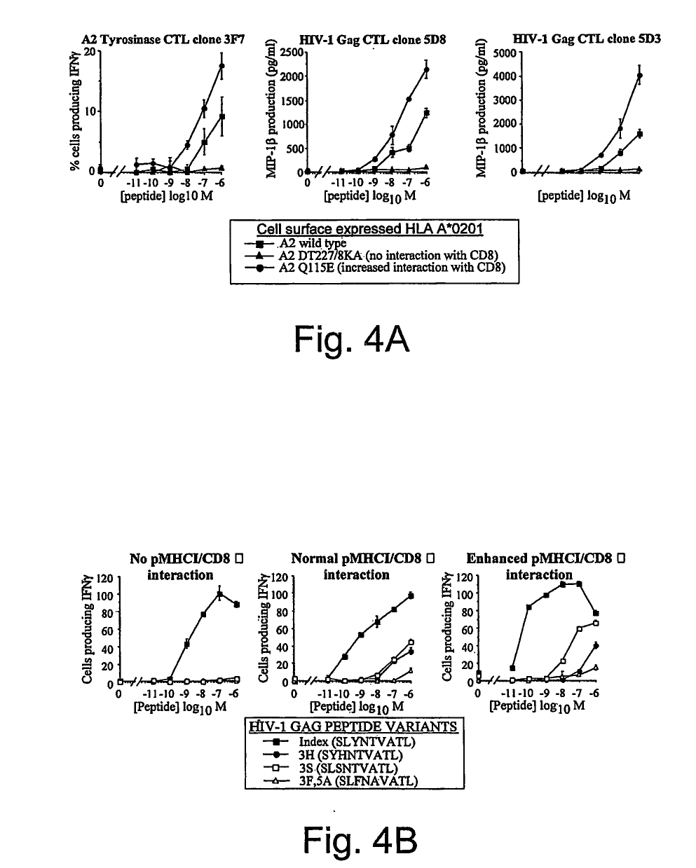

SEQ ID NO. 7 is the peptide SLYNTVATL from the peptide-MHC I complex (see FIG. 4), derived from the HIV-1 gag protein.

SEQ ID NO. 8 is the truncated HLA A peptide proposed by Collins et al. 1995 The three-dimensional structure of a class I major histocompatibility complex molecule missing the alpha 3 domain of the heavy chain. Proc Natl Acad Sci USA. 92:1218-21.

SEQ ID NO. 9 is the proposed alpha 3 domain of the HLA A peptide. Collins (supra) defines the domain boundary between residue 179 and 180 according to SEQ ID NO. 8.

SEQ ID NO. 10 is the peptide YMDGTMSQV derived from Tyrosinase.

SEQ ID NO. 11 is the peptide ITKGLGISYGR derived from the HIV-1 Tat protein.

SEQ ID NOS. 12-14 are HIV-1 Gag-derived peptide variants of SLYNTVATL (SEQ ID NO. 7), see FIG. 4.

The antigen presentation molecule is preferably an MHC Class I molecule and, in particular, is encoded by the human MHC Class I gene cluster. No allele is particularly preferred, as the CD8 coreceptor binding region of the MHC Class I molecule, the α3 domain, is highly conserved between MHCI proteins, although differences in the protein sequence do exist, for instance in the alleles HLA-A68 and HLA-B48.

The antigen presentation molecule is preferably produced by recombinant means, such as in a bacterial host, for example E. coli, or human cell lines, as is well known in the art. However, it is also envisaged that the molecule may be produced recombinantly in plants or fungi, or even in vitro, or completely synthetically.

It has been previously demonstrated that mutation of the α3 domain does not affect the binding of the TCR to the α1/α2 peptide-binding platform of MHC class I molecules [Purbhoo, M. A., et al 2001. J. Biol. Chem. 276, no. 35:32786]. However, in a preferred embodiment the avidity of the binding reaction between the α3 domain and the CD8 coreceptor is slightly strengthened.

A preferred method of genetic alteration of the antigen presentation molecule is described in the Examples. Essentially, using computer modelling techniques, the protein sequence of the antigen presentation molecule was altered so that the CD8 coreceptor was bound with greater avidity. The genetic sequence encoding this altered the protein sequence was then deduced, in order that the new antigen presentation molecule could be expressed.

Suitable methods for sequence modification are well known in the art, and include substitution, deletion, insertion, inversion and reversal, although it is generally sufficient simply to effect a point mutation.

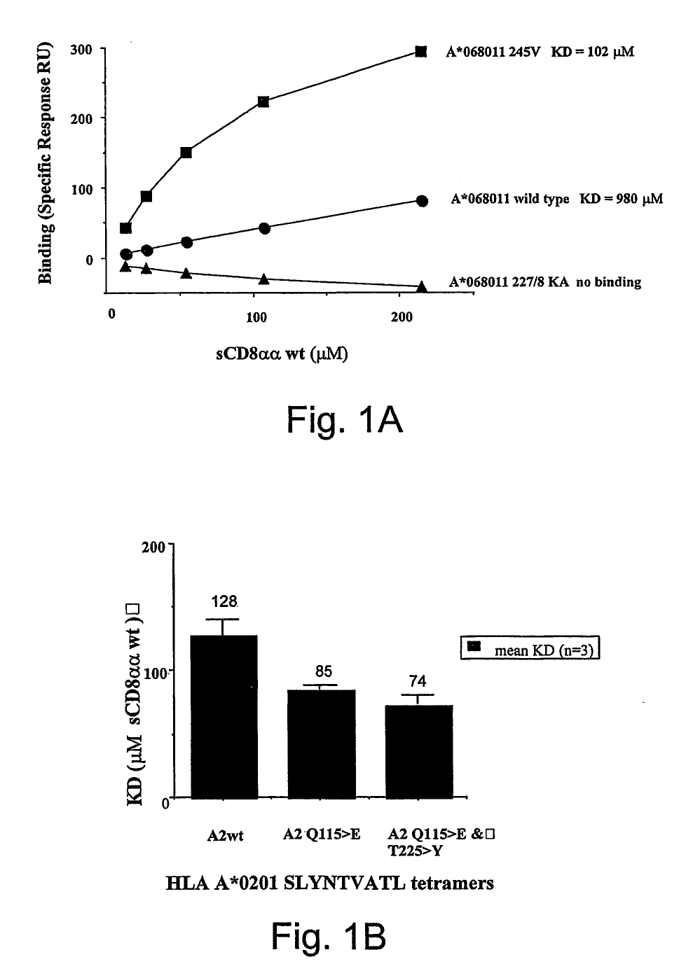

FIG. 1A shows surface plasmon resonance-measured binding of CD8 to wild type HLA A*6801, 227/8KA mutated HLA A*06801, and the 245A substituted HLA A*06801. The 245A substitution in HLA A*6801 increases the interaction between the A68 antigen presentation molecule and CD8 by almost 10 fold.

Binding was measured with the gene product (protein) rather than the gene (allele) itself. Any single base pair change in the gene, even one outside the open reading frame or one that does not alter the expressed protein, constitutes a different allele. Such differences are common place within the MHC locus which is the most variable part of the human genome.

Furthermore, FIG. 1B shows that the single substituted mutant Q115E and the double substituted mutant Q115E+T225Y both increase the interaction of the antigen presentation molecule HLA A*0201 with CD8.

In a preferred embodiment, it is preferred that the antigen presentation molecule of the invention is a substantially human MHC Class I molecule and comprises one of the following amino acids: 245A, 115E and 225Y. Combinations of these residues are also envisaged, although slightly less preferred, as they can lead to too great an avidity for CD8. Thus, a preferred molecule of the invention consists essentially of a human MHC Class I molecule substituted at one of positions 245, 115 and 225 with 245A, 115E or 225V, respectively. It will be appreciated that molecules of the invention also include fusion proteins and expression proteins comprising such a molecule, and that deletion mutants having essentially the same biological properties as the preferred molecules are also envisaged.

It will be appreciated that only that amount of MHC I that is needed to bind TCR, to antigen where necessary, and to CD8, is required. The remainder of the molecule, where not necessary for conformational stability, for example, is optional, and may be included, excluded, substituted and generally amended as desired and convenient, for example.

Although the above alterations are preferred, it will be appreciated that the present invention extends to any alteration, such as a deletion, insertion or substitution of one or a plurality of amino acids in the α3 domain that gives rise to an increase in the avidity of the interaction between the CD8 coreceptor and the MHC Class I antigen presentation molecule.

It is preferred that modifications are done in such a manner as to minimise the disruption to protein folding of the antigen presentation molecule, so as to not to disrupt the binding to CD8 or to disrupt other parts of the antigen presentation molecule, such as the peptide binding region. It is also preferred that any number of alterations may be made, provided that the antigen presentation molecule still provides a stimulatory or low level boosting effect on the immune system. It is not necessary that the antigen presenting molecule of the invention bind the TCR with exactly the same avidity as the wild type, provided that an effector response can be elicited.

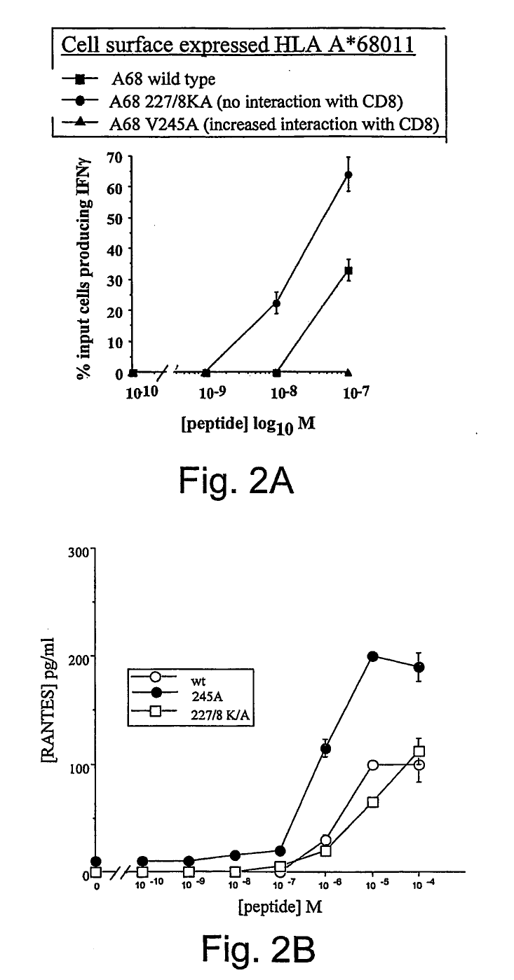

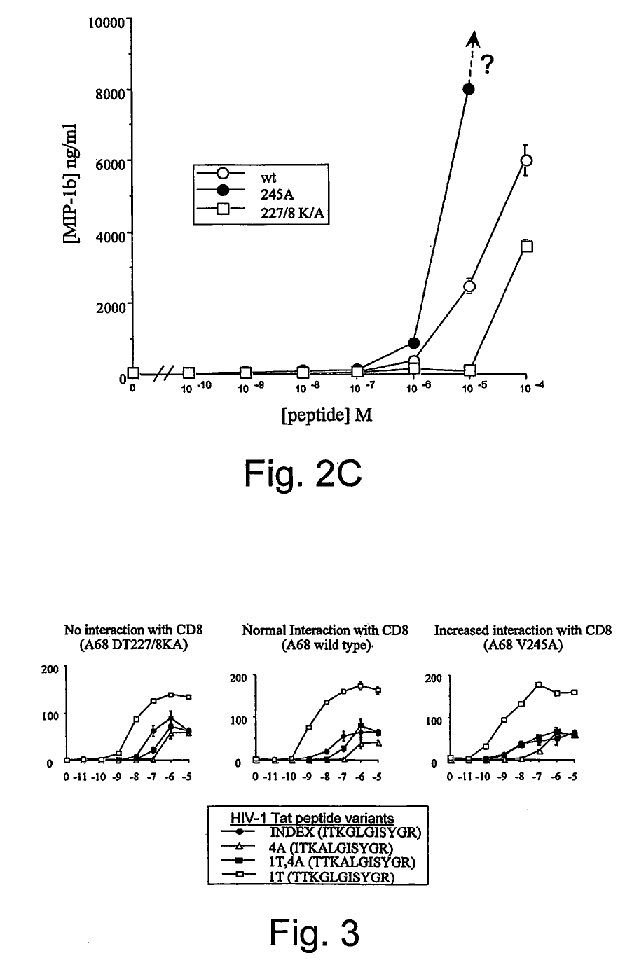

The stimulatory or low level boosting effect on the immune system seen with the present invention in FIG. 2, for example, arises due to, and can be measured by, the increased phosphorylation of the TCR ζ chain and LAT. Increased release of the chemokines MIP1β (a leukocyte-activating agent) and RANTES (a chemotactic agent responsible for attracting leukocytes) by CTL's bound by the present invention show that the altered pMHCI/CD8 interaction has led to enhanced antigen-sensitivity of the antigen-specific CTL.

The antigenic peptide is preferably complexed with, or bound to, the antigen presentation molecule, for instance at the peptide binding region. However, it is also envisaged that the peptide may not be complexed or bound to the antigen presentation molecule. It is envisaged that the antigen presentation molecule may be retained in the same conformation that it assumes when bound to the peptide, so that the antigen presentation molecule can activate the TCR in the absence of peptide. However, although the presence or absence of peptide may not affect the binding of CD8 by MHC I, it is generally preferred that the pMHC I complex is formed, as the TCR also needs to interact with the pMHC I complex.

The nature of the antigenic peptide may be any that is recognised as non-self or that is presented as part of the endogenous pathway. Accordingly, the peptide may be a peptide derived from a virally-encoded peptide or protein, or derived from a dysfunctional or dysregulated autologous peptide or protein. In particular, it is preferred that the peptide forming the pMHC I complex is derived from a tumour-associated peptide or protein, so that the administration of the present invention leads to the boosting of an anti-tumour response in the individual to which it is administered.

Preferred antigens of the invention may be from any tumour or disease causing organism, such as HIV.

The antigen presenting molecule of the invention may be used in a method to increase immune response to a selected disease state, and may suitably be administered in a carrier. Where the antigen presenting molecule is soluble, it may be sufficient to employ saline, and it is generally preferred to employ adjuvants, if desired, isotonicity agents, sterilants, buffers and other substances recognised in the art for the chosen administration form. Suitable administration forms are generally by injection, such as intravenous, intramuscular, intraperitoneal and subcutaneous, but may also be by any other suitable route.

In general, it is envisaged that molecules of the present invention will be used in prophylaxis or therapy with the aim of enhancing or stimulating an immune response. In such cases, the immune response is stimulated via the CTL, and is antigen specific. The antigen may generally be weak, or the patient may have a weak response, or be expected to have a weak response, to the antigen. Indeed, there may be no detectable response at normal challenge levels. The molecule of the invention for use under such circumstances will have an increased avidity for CD8, but not to such a level as to be approaching as strong as the TCR/pMHCI interaction. Such levels of interaction are referred to herein as being slightly increased levels.

However, the present invention also extends to molecules increases in avidity up to, and greater than, those associated with the TCR/pMHCI interaction, such as are observed with the A2Kb mutants, for example. Such molecules are pan activators of MHC Class I CTL, and appear to result in a complete loss of specificity. Use in vivo will often be contraindicated, lest an autoimmune condition be exacerbated, for example, but there may be occasions when this population can usefully be activated, such as in an acutely ill patient. In general, though, such pan activators are useful in in vitro situations, such as where it may be desired to investigate the effect of T cell activation.

The molecule of the invention, where not already associated with antigen, or a suitable antigenic fragment thereof, may be mixed with the antigen at any time, even after administration, although this is less preferred, as other antigens may then become associated with the molecule. A simple preparation of the antigen may be mixed with a preparation of the molecule, preferably in stoichiometric amounts, or with an excess of antigen. It is preferred that each be in a suitable vehicle, and it is further preferred that the vehicles be readily miscible, in order that admixture be facilitated. Advantageously, the mix of molecule and antigen is in a form ready for administration.

Similar considerations apply to pre-mixes and antigen/molecule hybrids, as appropriate.

In a preferred embodiment, there is provided a method for boosting a low-level immune response in an individual by administering a molecule of the invention to a patient in need thereof. There is also provided the use of a molecule of the invention in the manufacture of a medicament for the treatment or prophylaxis of a condition where the immune response is low. Such a low level may be judged by the skilled physician, and may range from being otherwise unable to raise a detectable response to simply wishing to achieve a response in faster time than would otherwise be achieved by use of a booster, for example.

The invention further provides a method for activating, or enhancing the activation level of a population of cytotoxic T lymphocytes specific for a particular antigen, in a mammal, said lymphocytes expressing the CD8 coreceptor, said method comprising administering an effective amount of an antigen presentation molecule having increased avidity for the CD8 coreceptor, compared to the molecule encoded by SEQ ID NO. 1, in vivo.

It will be appreciated that the method applies to any mammal, and may be used to stimulate a response to a perceived threat, as well as actual treatment of a disease state. Accordingly, the method is equally useful for both prophylaxis and therapy.

Any mammal may be treated, and it will be appreciated that, for non-human mammals, the equivalent coreceptor for CD8 is the target for the antigen presentation molecule. Suitable mammals include, apes, such as chimpanzees and gorillas, monkeys, horses, cats, dogs, pigs and generally other farm animals and pet animals.

The dosage may depend on the weight and age of the patient, and such parameters are within the general skill of the physician to determine and apply. Levels of MHC I and antigen are generally those suitable to elicit an immune response, or to boost such a response, and are readily determined by the skilled physician or veterinarian.

The present invention will now be further illustrated by the following, non-limiting Examples.

### EXAMPLES

### Example 1

Initial coordinates were taken from the crystal structure of the complex between human MHC class I HLA-A2 and the T cell co-receptor CD8αα solved at 2.65 Å resolution and deposited in the Protein Data Bank (Berman et al. Nucleic Acids Res. 2000, 28, 235-242), under the name 1 akj (Gao et al. 1997, Nature, 387, 630-634). Molecular Dynamics (MD) simulations were performed using CHARMM (version 27) (Brooks et al. 1983, J. Comp. Chem. 4, 187-217) and the standard all-atom parameter set (MacKerell et al. 1998, J. Phys. Chem. B, 102, 3586-3616). Hydrogens were added using the HBUILD module in CHARMM. Water molecules were added to the complex by superimposing a 16 Å sphere of TIP3P water molecules. The water molecules and the protein hydrogens were minimized and then equilibrated by a Molecular Dynamics (MD) simulation at 300 K for 5 ps, while keeping the remaining protein atoms fixed. The equilibration was performed using stochastic boundary conditions with a time step of 1 fs, a friction coefficient of 62 ps⁻¹ for the water oxygens and the SHAKE algorithm. The system was soaked again to fill any missing cavities with water. The solvent atom positions were optimized using 500 steps of steepest descents followed by 1000 steps of conjugate gradient. At the next step, the entire system was relaxed with 500 steps of steepest descents; this was switched to conjugate gradient until the convergence criteria of Root Mean Square Deviation (RMSD) gradient of the potential energy lower than 0.25 kcal/mol*Å had been achieved. A 14 Å non bonded cutoff, and a dielectric constant of ε=1 were employed. The system was simulated using stochastic boundary MD (Brooks et al. 1989, J. Mol. Biol. 208, 159-181). The system was divided into a 12 Å reaction region, a 4 Å buffer region and a reservoir. The friction coefficients for water oxygen and heavy atoms in the protein were 62 ps⁻¹ and 200 ps⁻¹, respectively (Brunger et al. 1987, Biochemistry, 26, 5153-5162). The relaxed system was equilibrated at 300K for 100 ps with a time step of 1 fs followed by 500 ps data collection with coordinates and energies saved to disk every 1 ps.

The mutants were designed using the multi-scale approach suggested be Glick et al (Glick et al. 2002, J. Am. Chem. Soc. 124, 2337-2344 and 2002, J. Med. Chem. 45, 4639-4646) where a hierarchy of models is generated using the k-means clustering algorithm for the potential binder (an amino acid side chain). The initial model is a single feature point located at the mean position of the side chain. The second model is formed by two points separated by a distance related to the dimensions of the side chain. The more feature points are added, the more detailed the model becomes.

Results

To design HLA-A2 heavy chain mutants with enhanced affinity for CD8αα we employed the multi-scale approach suggested by Glick et al (supra). We limited the algorithm to 4 feature points. The surface of the CD8αα protein that forms contacts with the HLA-A2 (Gao et al. supra) was systematically searched where amino acid side chains were used as “ligands”. Three models were selected for further MD study.

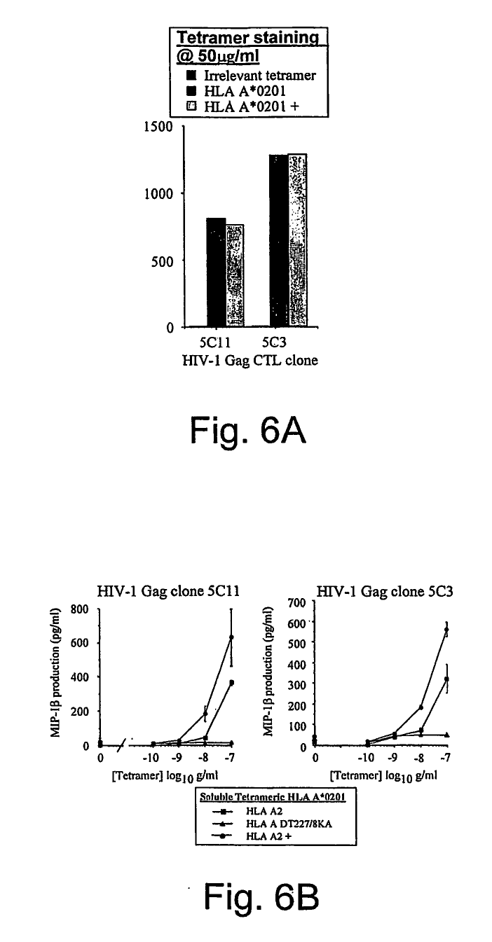

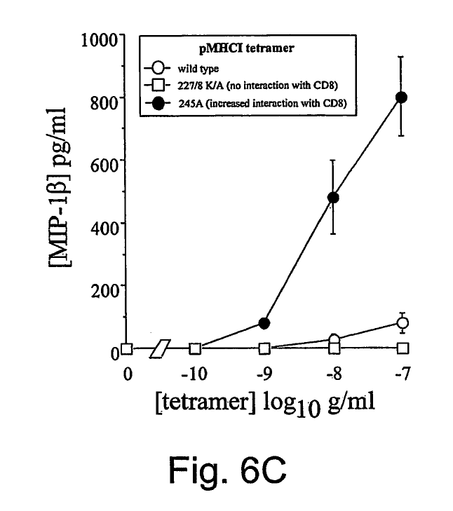

1. A single mutation of Q115 to E. The wildtype HLA-A2, Q115:Oε1 forms a weak H-bond interaction with CD8αα R4:Nη1. (The Oε1 . . . Nη1 distance in the crystal structure is 3.18 Å). This interaction was replaced by a shorter H-bond between HLA-A2 Q115E:Oε1 and CD8α1 R4:Nη1 as shown in FIG. 6. The MD simulation showed that the Oε1 . . . Nη1 average distance was 2.56 Å and fluctuated between 2.42 Å to 2.82 Å. This short distance also indicates that the Q115E carboxylate and R4 guanidinium moieties form a strong electrostatic interaction that is likely to increase the avidity between the two biomolecules.

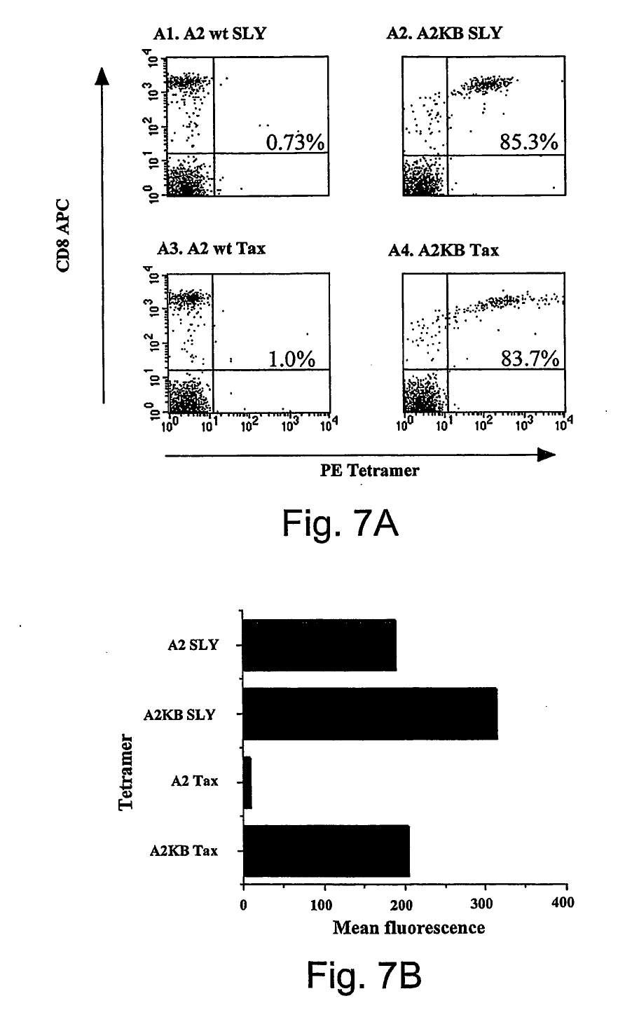

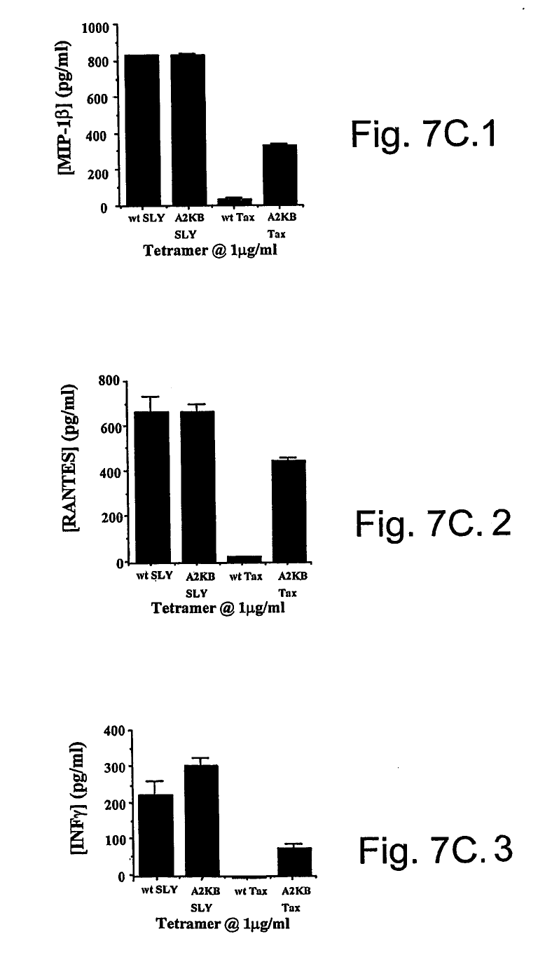

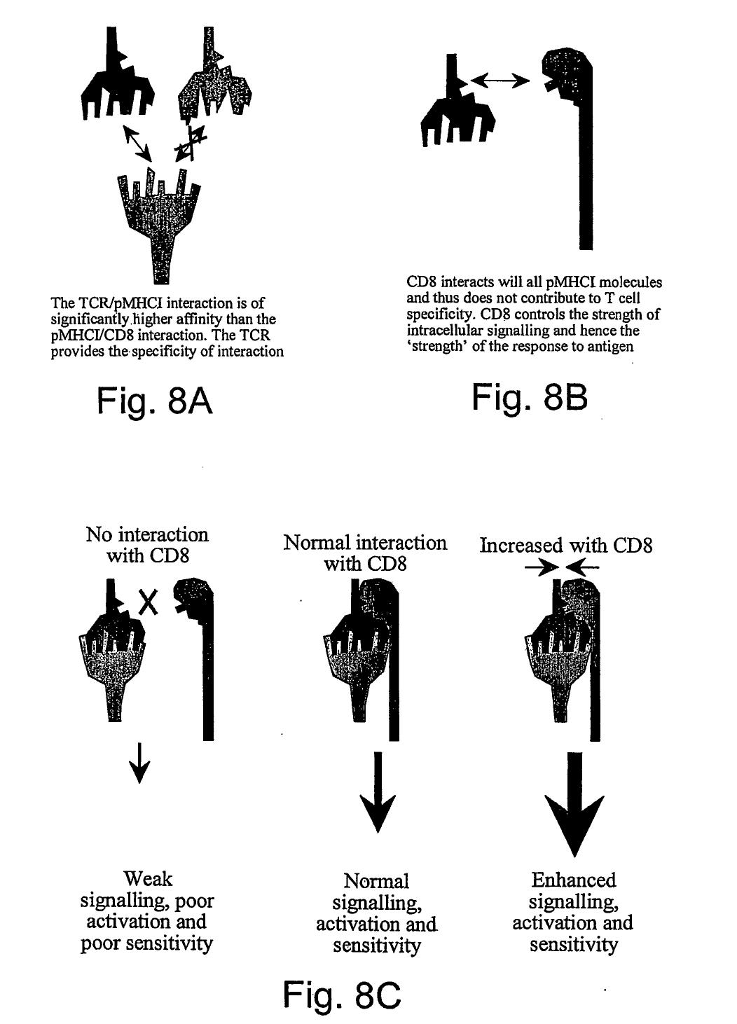

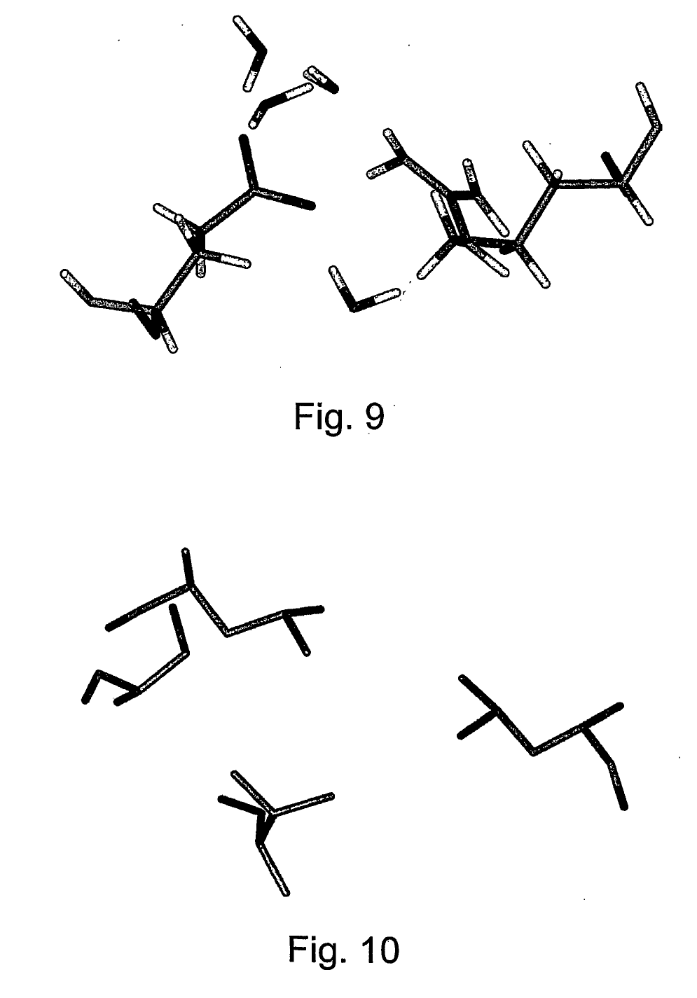

2. A single mutation of T225 to V. The crystal structure shows that the side chain of the wildtype T225 does not form any obvious interaction with the CD8αα. The MD trajectories indicate that the T225V hydrophobic side chain fills the hydrophobic pocket formed by the side chains of two L97 residues (one from each CD8 subunit) and Cβ of ser 31 (CD8α1) as shown in FIG. 7

3. Combined Q115E and T225V mutations. The interaction between Q115 and T225 is negligible since the distance between their Cα atoms in the crystal structure is 30.7 Å. Therefore, we utilised the combined double mutation in the expectation that it will have a higher affinity towards the CD8αα than any of the single mutations.

Thus, using the crystal structures of HLA A2 and the HLA A2/CD8 co-crystal structure, a panel of MHCI mutations predicted to increase the HLA A2/CD8 interaction were produced. The predicted mutations were engineered into biotinylated pMHCI monomers and tested by surface plasmon resonance for their ability to bind CD8. The strength of the HLA A2/CD8 interaction was in the range of CD8/MHCI interactions measured previously (K_{D}=135 μM). Introducing the HLA A⁺ mutation into MHCI was shown to significantly increase the pMHCI/CD8 interaction as predicted (K_{D}=85 μM) HLA A2 (DT227/8KA) showed no interaction with CD8 (K_{D}=>10,000 μM). To study the effect of increasing the pMHCI/CD8 interaction we introduced pMHCI mutations with abrogated, normal and enhanced pMHC/CD8 interaction into cell surface expressed HLA A2 and soluble tetrameric pMHCI reagents.

Discussion

The K_{D} for the HLA A2/CD8 wildtype is 130 μM. The Q115E mutation increased the affinity to K_{D}=80 μM. This result supports the hypothesis that the electrostatic and strong H-bond interactions between the guanidinium (CD8α1 R4) and the carboxylate (HLA-A2 Q115E) moieties indeed increased the avidity between the biomolecules.

Proteins with the mutation T225V did not appear to refold well. It is possible that the folding path of the double mutated protein is therefore different from the single mutated T225V protein. We, therefore, tried other mutations at this position. A T225 to Y substitution did produce a protein that refolded well and, in addition to the Q115E substitution mentioned above, appeared to further enhance CD8 binding (FIG. 1B). The double mutation Q115 and T225Y mutations yielded a K_{D} of 70 μM.

The manufacture of pMHCI monomer and the creation of multimeric forms (tetramers) is described in O'Callaghan et al. (O'Callaghan et al. 1999, Anal Biochem. 266, no. 1:9). pMHCI was multimerised by the addition of PEconjugated streptavidin. This is expanded on in our recent JBC paper (Purbhoo et al. 2001, J. Biol. Chem. 276, no. 35:32786), incorporated herein by reference, where we also describe how C1R cells expressing wild type and mutant pMHCI are manufactured.

### Example 2

Results and Discussion

Increasing the MHC/Coreceptor Interaction

Mutations that might increase this interaction were predicted by molecular modelling based on the structures of HLA A2, HLA A68 and the HLA A2/CD8 cocrystal (Gao et al. 1997, Nature 387, no. 6633:630), as described in Example 1 above. These mutations were engineered into biotinylated pMHCI molecules as previously described (Purbhoo et al. 2001, J Biol Chem 276, no. 35:32786). Mutant pMHCI molecules were then tested for their ability to bind to soluble CD8αα by surface plasmon resonance using techniques described previously (Purbhoo et al., supra). We have thus far determined that mutation of position 245 in HLA A*68011 from valine to alanine increases the binding to CD8αα by almost 10 fold (FIG. 1A). Surface plasmon resonance shows that mutation of Q115->E and and a double substitution of Q115->E and T225->Y increase the interaction of HLA A*0201 with CD8αα (FIG. 1B). With few exceptions (eg. HLA A68 and HLA B48), the α3 domain of all classical MHC class I molecules (those that bind peptides and present them to CTL) is identical in sequence to HLA A*0201. Consequently, these mutations are expected to increase the CD8-binding of any MHC class I molecule.

The Effects of Enhanced pMHCI/CD8 Interaction

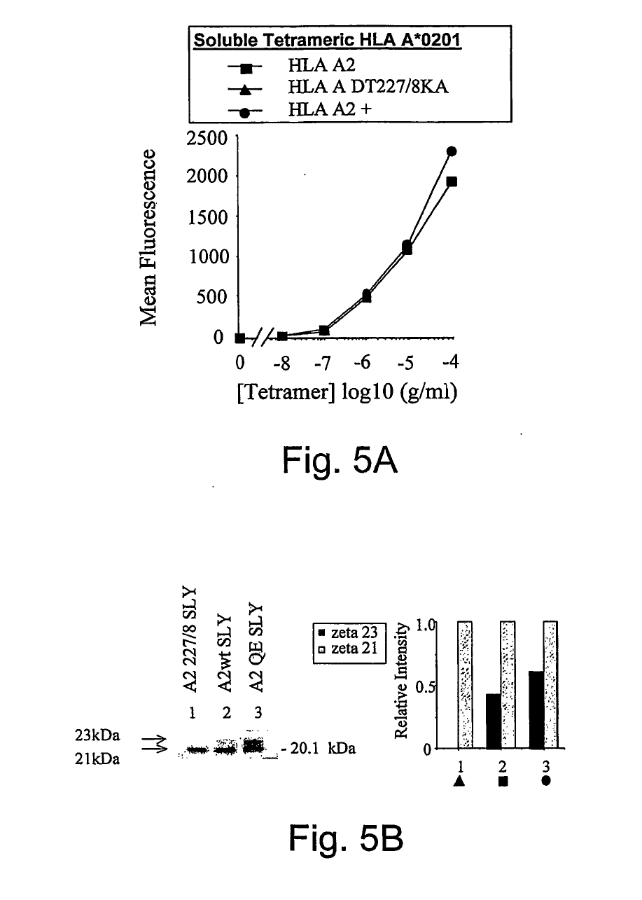

Presentation of antigen on the surface of target cells in the context of increased pMHCI/CD8 interaction significantly enhances their sensitivity to antigen in interferon γ ELISpot assays (FIGS. 2A & 4A). Increased pMHCI/CD8 interaction also enhances the antigen-sensitivity of antigen-specific CTL as measured by the release of the β-chemokines MIP1β (FIGS. 2B & 4A) and RANTES (FIG. 2C). The presentation of cells expressing MHC class I with increased pMHCI/CD8 interaction does not result in the spontaneous activation of CTL in the absence of antigen (FIGS. 2, 3 & 4). This finding rules out the possibility that increasing the pMHCI/CD8 interaction to a KD of 85 μM MHCI/coreceptor interaction results in the recognition of self antigen. To further test for any alteration in the specificity of CTL we used altered peptide ligands (APL) that differed by just one or two amino acids from the agonist peptide. Increased pMHCI/CD8 interaction did not result an altered pattern of ligand-recognition (FIGS. 3 & 4B). Increased pMHCI/CD8 interaction was also able to enhance responses to weak agonist ligands (FIGS. 3 & 4B). Examination of early intracellular tyrosine phosphorylation events indicates that increase pMHCI/CD8 interaction results in increased tyrosine phospholylation of the TCR ζ chain (FIGS. 5A & 5B). It thus appears that while the TCR provides the exquisite specificity of the T cell it is the coreceptor that determines the outcome of this interaction. Increasing the pMHCI/CD8 interaction to a KD of 85 μM does not affect T cell specificity but does increase both their sensitivity to antigen (by over tenfold) and the quantity of an effector function in response to a given quantity of antigen.

### Example 3

The Benefits of Increased MHC/Coreceptor Interaction Extend to Soluble Forms of Antigen

Current models of T cell activation propose that complete activation requires two signals, one provided via the TCR and the other via accessory molecules such as CD28 on the T cell surface (Schwartz, 1990, Science 248, no. 4961:1349 and Lenschow et al. 1996, Annu. Rev. Immunol. 14:233). Most of the work leading to such models has been with CD4+ Th cells (reviewed in June et al. 1990, Immunol. Today 11, no. 6:211 and Janeway et al. 1994, Cell 76, no. 2:275 and Linsley et al. 1993, Annu. Rev. Immunol. 11:191). While such a model may still hold for CD4+ cells it is becoming increasingly challenged as model for the activation of CD8+ CTL. First, it seems to be unlikely to us that such a model should hold when effector CTL kill cells infected with intracellular pathogens or tumour cells. Only bone-marrow derived cells bear ligands for CD28 (B7.1 and B7.2). The requirement of such molecules in the activation of effector CTL would purport that CTL would be unable to eliminate non-bone-marrow-derived virally infected or tumour cells. The advent of soluble multimerised pMHCI complexes has enabled a direct assessment of the requirement of a ‘second’ signal. We have used this technology to show that multimerised, soluble pMHCI can deliver a normal pattern of early intracellular signalling to effector CTL that results in the activation of effector functions (Purbhoo et al. 2001, J. Biol. Chem. 276, no. 35:32786). Other groups have gone further with this technology and shown that the proliferation and differentiation of naive CD8+ T cells into cytotoxic effector cells does not require costimulation (Wang et al. 2000, J. Immunol, 164, no. 3:1216). Thus it appears likely that soluble pMHC I alone is sufficient to deliver a complete activation signal to MHC class I-restricted T cells.

We have manufactured soluble versions of pMHCI with increased affinity for the CD8 coreceptor using the mutated MHC class I molecules described above. We observe that these molecules deliver an enhanced signal to specific CTL and result in similar increases in CTL activation as those when the antigen is delivered on the surface of a target cell (FIG. 6). This result paves the way for the use of soluble altered antigen in vivo to kick start antigen-specific CTL responses.

Conclusions

These results show that the antigen presenting molecule of the invention improves T cell immunity and makes T cells more sensitive to antigen. This makes T cells more effective in response to a given quantity of antigen. The results also show that the antigen presenting molecule of the invention is effective as a soluble molecule.

Injection of soluble forms of tumour antigen containing the antigen presenting molecule of the invention may significantly improve immunity to tumours by signalling anti-tumour CTL to proliferate. Adoptive transfer experiments in mice have established a correlation between the number of anti-tumour CTL and tumour clearance. This invention could further be used to develop more sensitive technologies for the detection of T cell responses.

### Example 4

Development of Novel MHC Class I Specific Superantigens

Wild type and mutant tetramers folded around either the SLYNTVATL peptide (HIV Gag) or LLFGYPVYV peptide (HTLV-1) were produced. In FIG. 7A we isolated fresh PBMC from a HLA A2 positive normal donor by density gradient separation. 2.5×10⁵ PBMCs per stain were washed and resuspended in Facs buffer (2% FCS/PBS) then stained with either 1 μg of A1. A2 SLYNTVATL, A2. A2 Kb SLYNTVATL, A3. A2 LLFGYPVYV or A4. A2 A2 Kb LLFGYPVYV phycoerythrin conjugated tetramer for 20 mins at 37° C. Each sample was subsequently stained with anti-CD8 APC and anti-CD3 PerCP, washed twice, resuspended in FACS buffer then analysed on a FACScalibur. Analysis was carried out, gating on the live lymphocyte gate and CD3⁺ cells only. pMHCI tetramer folded around SLYNTVATL or LLFGYPVYV peptide stained 0.73% and 1% of CD3+ CD8+ PBMC respectively. Introducing the kb mutation into pMHCI tetramers folded around SLYNTVATL or LLFGYPVYV peptide increases this staining to 85.3% and 83.73% respectively. Therefore increasing the affinity of the CD8/pMHCI interaction by approximately 25 fold, so that its strength approaches, or exceeds, that of the TCR/pMHCI interaction, results in a total loss of tetramer staining specificity.

FIG. 7A shows that the human pMHCI/CD8 interaction is characterised by low affinity (K_{D}=128 μM). We have produced pMHCI with an extremely high affinity for CD8 (K_{D}=<5 μM) by using the murine α3 domain in place of the human.

003 is the immunodominant CTL clone from a HIV-1 patient (epitope SLYNTVATL: SEQ ID NO. 7). We have previously sequenced proviral DNA of the A2 restricted Gag p17-18 epitope from patients mounting a CTL response to this epitope and identified a panel of naturally occurring variants with different effects on recognition by the 003 CTL clone. The index peptide acts as full agonist whereas 3H and 3S are weak agonists and 3F,5A a CTL antagonist. IFNγ ELISPOT demonstrates that increasing the pMHCI/CD8 interaction enhances CTL sensitivity to the index peptide and to a lesser degree to the weak agonists (3H and 3S). However presentation of an antagonist in the context of the HLA A+ mutant (K_{D}=85 μM) does not result in CTL activation. SLYNTVATL (SEQ ID NO. 7) is the Index peptide, SYHNTVATL (SEQ ID NO. 12) is the so-called 3H peptide, SLSNTVATL (SEQ ID NO. 13) is the so-called 3S peptide, and SLFNAVATL (SEQ ID NO. 14) is the so-called 3F,5A peptide. The numbering and lettering referring to the amino acid position, and identity of, the variant amino acid, compared to SLYNTVATL (SEQ ID NO. 7). Thus, anti-tumour and anti-viral CTL antigen sensitivity and affectivity is enhanced by increasing the HLA A2/CD8 interaction without any loss of specificity.

In FIG. 7B, 1×10⁵ 003 CTL (HIV Gag clone, specific epitope SLYNTVATL) were washed and resuspended in 20 μl pf PBS and stained with 1 μg of either A2 SLYNTVATL, A2 Kb SLYNTVATL, A2 LLFGYPVYV or A2 Kb LLFGYPVYV phycoerythrin conjugated tetramer for 20 mins at 37° C. Samples were then washed twice and resuspended in PBS and analysed using a FACScalibur flow cytometer. Wild type MHCI tetramer folded around the SLYNTVATL peptide stained 003 CTL well, whereas wild type pMHCI tetramer folded around an irrelevant Tax peptide (LLFGYPVYV) did not stain. Therefore, pMHCI tetramers with ‘normal’ pMHCI/CD8 interaction stain CTL specifically. pMHCI tetramers folded around SLYNTVATL or irrelevant peptide will both stain 003 CTL if the A2 Kb mutation is introduced. The A2 Kb mutation increases the pMHCI/CD8 interaction by 25 fold. Therefore, increasing this interaction gives a reagent that will stain all CTL independently of a specific TCR/pMHCI interaction. Data with a panel of other anti-viral CTL clones shows the same effect (data not shown).

In FIG. 7C, 5×10⁴ 003 CTL (HIV Gag specific clone, epitope SLYNTVATL) were activated with either A2 SLYNTVATL, A2 A2 Kb SLYNTVATL, A2 LLFGYPVYV or A2 A2 Kb LLFGYPVYV Phycoerythrin conjugated tetramer at 1 μg/ml. After 4 hours at 37° C. the supernatant was removed and analysed for MIP-1β, RANTES and IFNγ by ELISA. A2 SLYNTVATL and A2 M SLYNTVATL tetramer activated 003 CTL well, producing all 3 lymphokines. A2 LLFGYPVYV did not activate 003 CTL, therefore, CTL activation by tetramer with ‘normal’ pMHCI/CD8 interaction maintains specificity. Notably, A2 A2 Kb LLFGYPVYV resulted in significant production of MIP-1β, RANTES and IFNγ above background, which suggests that increasing the pMHCI/CD8 interaction so that it becomes greater than the TCR/pMHCI interaction can result in CTL activation in the absence of specific peptide recognition.

The pMHCI/CD8 interaction is significantly weaker than the TCR/pMHCI interaction (up to 100×) (Wyer, et al., Immunity, 1999, 10:219-225; Gao, et al., J Biol Chem, 2000, 275:15232-15238). In the previous Examples, we used MHCI mutations to increase the pMHCI/CD8 interaction by factors of between 2-10 fold and not higher than K_{D}=70 μM but, despite this, the strength of the pMHCI/CD8 still remained significantly less than the TCR/pMHCI interaction. As a result, the TCR/pMHCI interaction dominates, and the specificity of the CTL response is maintained. A completely different effect on CTL activation is seen if the pMHCI/CD8 interaction is increased by 25 fold, so that the strength of this interaction actually begins to approach, or exceed, the strength of the TCR/pMHCI interaction. In the case of CTL with weak TCR/pMHCI interaction, then the pMHCI/CD8 may actually exceed it.

We previously demonstrated that mouse class I interacts with human CD8αα with K_{D} of 18 μM (Purbhoo, et al., J Biol Chem, 2001, 276:32786-32792). If the α3 domain of human HLA A2 is substituted with the α3 domain of murine H-2K^{b}, thereby producing a chimeric class I, designated A2Kb, then the pMHCI/CD8 interaction is increased to a K_{D} of 5 μM (Man-Lik Choi, et al., Journal of Immunology, 2003, 171:5116-5123; Choi, et al., J Immunol Methods, 2002, 268:35-41). pMHCI tetrameric reagents folded around specific epitopes display exquisite specificity for CTL that bear the T cell receptor that sees the specific peptide/HLA combination (Altman, et al., Science, 1996, 274:94; Burrows, et al., J Immunol, 2000, 165:6229-6234).

Increasing the pMHCI/CD8 interaction to a K_{D} of 5 μM produces pMHCI tetrameric reagents that will stain any CD8+ T cell. in PBMCs and any CD8+ CTL clone, irrespective of the peptide in the MHCI binding groove (FIGS. 7A&B). A2 Kb pMHCI tetrameric reagents have the ability to activate CTL of any peptide specificity resulting in the production of IFNγ, RANTES and MIP-1β (FIG. 7C). Thus, by increasing the pMHCI/CD8 interaction by 25 fold, we produced a novel class I superantigen capable of activating any CD8+ T cell. Activation is mediated via the pMHCI/CD8 interaction alone, in the absence of a specific TCR/pMHCI interaction.

Conclusions

T cells are one of the main cellular components of the adaptive immune response. T cells can be divided into T helper cells (Th) and Cytotoxic T Lymphocytes (CTLs). Th cells are CD4⁺, and recognise peptides from exogenous proteins that are presented in the context of MHC class II. Th cells produce cytokines which can: Help B cells (antibody production); stimulate macrophages (phagocytosis); and, help CTL. CTL recognise foreign peptides (8-11 amino acids long) from endogenous proteins in the context of MHC class I molecules. CTL can eliminate: virally infected cells and tumour cells. Therefore, CD4⁺ and CD8⁺ T cells have very distinct functional roles. An extremely strong pMHCI/CD8 interaction can be used to produce class I superantigens capable of activating CD8⁺ T cells, regardless of their antigen specificity. Bacterial superantigens often activate both CD4+ and CD8+ T cells, as well as other cellular components of the immune system. However, in situations where it is desirable to activate CD8⁺ T cells only, then the antigens described in this Example are extremely useful. It has already been demonstrated that bacterial superantigens can reactivate antigen specific CD8⁺ memory cells, and have protective properties in vivo against lethal viral infections (Coppola, et al., Int Immunol, 1997, 9:1393-1403; Okamoto, et al., Infect Immun, 2001, 69:6633-6642).

1-31. (canceled)

32. An antigen presenting molecule capable of activating a cytotoxic T lymphocyte by interaction with a T cell receptor and CD8 coreceptor thereon, wherein the antigen presentation molecule displays an increased avidity for the CD8 coreceptor, compared to the molecule encoded by SEQ ID NO. 1 in vivo.

33. A molecule according to claim 32, wherein at least a part thereof corresponds sufficiently closely to MHC I to be able to interact with both the T cell receptor and CD8 coreceptor.

34. A molecule according to claim 33, said molecule comprising MHC I domains or functional homologues thereof sufficient to enable interaction with both the T cell receptor and CD8 coreceptor.

35. A molecule according to claim 33, said molecule further comprising the antigen-binding domain of MHC I.

36. A molecule according to claim 33 which, apart from any difference necessary to enable the increased avidity, is humanised over at least 99% of its sequence.

37. A molecule according to claim 33 that is unable to activate the cytotoxic T cell in the absence of antigen or suitable fragment thereof.

38. A molecule according to claim 33 comprising the HLA A+ mutant.

39. A molecule according to claim 33, wherein the avidity of the molecule for CD8 has a K_{D} of between 60 μM and 120 μM.

40. A molecule according to claim 39, wherein the avidity is between 60 μM and 100 μM.

41. A molecule according to claim 33, wherein one or more of the amino acid residues 223-229 in the α3 domain loop is substituted.

42. A molecule according to claim 33, having the Q115E substitution.

43. A molecule according to claim 33, having the T225Y substitution.

44. A molecule according to claim 42 having an allelic MHC I sequence, other than one or both of the Q115E and T225Y substitutions.

45. A molecule according to claim 43 having an allelic MHC I sequence, other than one or both of the Q115 E and T225Y substitutions.

46. A molecule according to claim 33 which is HLA A*68011.

47. A molecule according to claim 33, having the same sequence as an allelic human MHC I molecule, but having the 245A substitution.

48. A molecule according to claim 33, bound to an antigen, or fragment thereof, such that the cytotoxic T cell is activatable thereby.

49. A molecule according to claim 48, wherein the antigen, or fragment thereof, is viral in origin.

50. A molecule according to claim 48, wherein the antigen, or fragment thereof, is cancerous in origin.

51. A molecule according to claim 49, wherein the virus is HIV, HTLV or EBV.

52. A molecule according to claim 49, wherein the antigen is Tat, Gag or Pol.

53. A molecule according to claim 32 which has an avidity for CD8<60 μM.

54. A molecule according to claim 53, wherein at least a majority of the human MHC I domain has been substituted with an equivalent amount of the mouse α3 domain.

55. A molecule according to claim 33 which is soluble.

56. A molecule according to claim 33 in the form of a tetramer.

57. A nucleotide expression vector encoding a molecule according to claim 33.

58. A host containing a vector according to claim 57.

59. A host according to claim 58, wherein said host is a deletion mutant engineered to express no other antigen presenting molecule.

60. A method for boosting a low-level immune response in an individual by administering a molecule according to claim 33 to a patient in need thereof.

61. A method for activating, or enhancing the activation level of a population of cytotoxic T lymphocytes specific for a particular antigen, in a mammal, said lymphocytes expressing the CD8 coreceptor, said method comprising administering an effective amount of an antigen presentation molecule having increased avidity for the CD8 coreceptor, compared to the molecule encoded by SEQ ID NO. 1 in vivo.


SEQUENCE LISTING (9 sequences)

SEQ ID NO: 1 - DNA, 1098 bp, Homo sapiens
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 2 - protein, 365 aa, Homo sapiens [sequence omitted]

SEQ ID NO: 3 - DNA, 1098 bp, Homo sapiens
atggccgtcatggcgccccgaaccctcgtcctgctactctcgggggccctggccctgacccagacctgggcgggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccggaacacacggaatgtgaaggcccagtcacagactgaccgagtggacctggggaccctgcgcggctactacaaccagagcgaggccggttctcacaccatccagatgatgtatggctgcgacgtggggtcggacgggcgcttcctccgcgggtaccggcaggacgcctacgacggcaaggattacatcgccctgaaagaggacctgcgctcttggaccgcggcggacatggcagctcagaccaccaagcacaagtgggaggcggcccatgtggcggagcagtggagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggtggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 4 - protein, 365 aa, Homo sapiens [sequence omitted]

SEQ ID NO: 5 - DNA, 1089 bp, Homo sapiens
atgctggtcatggcgccccgaaccgtcctcctgctgctctcggcggccctggccctgaccgagacctgggccggctcccactccatgaggtatttctacacctccgtgtcccggcccggccgcggggagccccgcttcatctcagtgggctacgtggacgacacccagttcgtgaggttcgacagcgacgccgcgagtccgagagaggagccgcgggcgccgtggatagagcaggaggggccggagtattgggaccgggagacacagatctccaagaccaacacacagacttaccgagagagcctgcggaacctgcgcggctactacaaccagagcgaggccgggtctcacaccctccagagcatgtacggctgcgacgtggggccggacgggcgcctcctccgcgggcataaccagtacgcctacgacggcaaggattacatcgccctgaacgaggacctgcgctcctggaccgccgcggacacggcggctcagatctcccagcgcaagttggaggcggcccgtgtggcggagcagctgagagcctacctggagggcgagtgcgtggagtggctccgcagatacctggagaacgggaaggacaagctggagcgcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtggacagctgtggtggtgccttctggagaagagcagagatacacatgccatgtacagcatgaggggctgccgaagcccctcaccctgagatgggagccgtcttcccagtccaccgtccccatcgtgggcattgttgctggcctggctgtcctagcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttga

SEQ ID NO: 6 - protein, 362 aa, Homo sapiens [sequence omitted]

SEQ ID NO: 7 - protein, 9 aa, Artificial Sequence
  peptide
SLYNTVATL

SEQ ID NO: 8 - protein, 275 aa, Homo sapiens [sequence omitted]

SEQ ID NO: 9 - protein, 96 aa, Homo sapiens
QRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE